(19) United States
(12) Patent Application Publication
(10) Pub. No.: US 2003/0162253 A1
(43) Pub. Date: Aug. 28, 2003

(54) Recombinant plasmid psp1, microorganisms transformed therewith, and method for producing an alkaline protease vapk

(76) Inventors: Ghee Hong Jin, Seoul (KR); Hyune Hwan Lee, Kyungkee-do (KR); Hyune Mo Rho, Seoul (KR); Hyung Seok Kim, Inchon (KR)

Correspondence Address: BURNS DOANE SWECKER & MATHIS L L P, POST OFFICE BOX 1404, ALEXANDRIA, VA, 22313-1404, US

(21) Appl. No.: 10/333,202

(22) PCT Filed: Jul. 19, 2001
(86) PCT No.: PCT/KR01/01231

(30) Foreign Application Priority Data
Jul. 19, 2000 (KR) 2000/41213

(51) Int. Cl. C12P 21/02; C12N 1/21; C12N 9/50; C12N 15/74

(52) U.S. Cl. 435/69.1; 435/219; 435/320.1; 435/252.33; 435/488

(57) ABSTRACT

The invention relates to recombinant plasmid pSP1 harboring the gene vapk, which encodes alkalic protease VapK, and par gene, which is associated with the stability of plasmid, a microorganism Vibrio metschnikovii transformed therewith, and method for producing an alkaline protease VapK using the same microorganism.

### TECHNICAL FIELD

The present invention relates to a recombinant plasmid vector pSP1 comprising the gene vapk encoding an alkaline protease VapK and the par gene increasing the stability of plasmid vector itself, a microorganism Vibrio metschnikovii transformed therewith, and a method for producing an alkaline protease VapK using the same microorganism.

### BACKGROUND ART

In general, an alkaline protease derived from Vibrio sp. is referred to as an enzyme that degrades proteins into amino acids and small peptides, and many microorganisms, such as Bacillus sp. and Serratia sp., are well-known to excrete alkaline protease

The most important factor in producing alkaline protease using the said microorganisms is the high productivity of alkaline protease, which affects the costs of producing process.

According to the recent studies, recombinant vectors comprising the gene encoding alkaline protease were prepared using genetic engineering techniques and transformants therewith were also prepared in order to raise the productivity of alkaline protease. In addition, since the stability of the plasmid vector may directly affect the productivity of the alkaline protease, several studies related to the improvement of stability of recombinant plasmid vectors have been reported.

The present inventors previously filed a patent application (KR 1999-12588) titled “alkaline protease VapK suitable for laundry detergent, vapk gene, recombinant expression vector, and transformed microorganism”, in which the microorganism is Vibrio metschnikovii KS1 and the recombinant expression vector is pSBCm. The same microorganism and recombinant expression vector are also used in the present invention.

The present inventors have studied and tested intensively and extensively in order to produce the transformed microorganism with the recombinant plasmid vector with improved stability. After all, it was prepared a recombinant plasmid vector comprising the alkaline protease gene, isolated from Vibrio metschnikovii KS1 and the par gene with the function of increasing the stability of plasmid vector itself, and transformed Vibrio metschnikovii with the said recombinant plasmid vector, thereby the transformed microorganism comprising plasmid vector having improved stability as well as improved protease activity was also prepared.

### SUMMARY OF THE INVENTION

The present invention provides a recombinant plasmid vector pSP1 in which the par gene is cloned into a recombinant plasmid vector pSBCm (KCCM-10142) comprising the vapk gene encoding an alkaline protease to improve the stability of plasmid vector itself.

The present invention also provides a method for producing the recombinant plasmid vector pSP1 of claim 1, by inserting the par gene into between PvuII and HincII recognition sites of a recombinant plasmid vector pSBCm (KCCM-10142) comprising the vapk gene encoding an alkaline protease derived from Vibrio sp., wherein the par gene has the function of improving the stability of the plasmid vector itself.

Also, the present invention provides a transformed host cell comprising the recombinant plasmid vector pSP1.

Furthermore, the present invention provides a transformed host cell, where the host cell comprises Escherichia coli or Vibrio metschnikovii.

The present invention also provides a method for producing an alkaline protease VapK using the transformed host cell mentioned above.

### BRIEF DESCRIPTION OF DRAWINGS

Hereinafter, the present invention is described in detail by referring to the attached drawings.

FIG. 1 illustrates the result of electrophoresis of the recombinant plasmid vector pSBCm comprising the vapk gene encoding an alkaline protease VapK. Line M represents size markers, line 1 represents a recombinant plasmid vector pSBCm, and line 2 represents a recombinant plasmid vector pSBCm/Hind III consisting of a vehicle vector pKF3 of 2.2 kb and a Vibrio alkaline protease gene of 2.9 kb.

FIG. 2 illustrates the restriction enzyme map of the recombinant plasmid vector pSBCm comprising the alkaline protease gene vapk.

FIG. 3 illustrates the restriction enzyme map of the recombinant plasmid vector pSP1 comprising the alkaline protease gene vapk and the par gene.

### DETAILED DESCRIPTION OF THE INVENTION

The present invention provides a microorganism transformed with the recombinant plasmid vector comprising the gene vapk encoding an alkaline protease VapK and the par gene improving the stability of plasmid vector itself, and a method for improving the stability of the recombinant plasmid vector within the transformed microorganism mentioned above.

According to the present invention, the gene encoding the alkaline protease is isolated by the following steps: culturing Vibrio metchnikovii KS1, centrifuging and recovering the cells of Vibrio metchnikovii KS1, disrupturing the cells and centrifuging them to obtain the cell-free supernatant, and extracting the chromosomal DNA of Vibrio metchnikovii KS1 from the cell-free supernatant. Using the said chromosomal DNA of Vibrio metchnikovii KS1, the transformed Escherichia coli was produced, that is, by digesting the said chromosomal DNA of Vibrio metchnikovii KS1 with restriction enzymes like Hind III to produce DNA fragment, cloning the DNA fragment into plasmid vectors to construct pSBCm and pSP1, and transforming them into E. coli.

First, the expression of the alkaline protease was conducted by using the above recombinant E. coli comprising plasmid vector pSBCm, and the expression level of the alkaline protease produced from the transformed E. coli under alkaline condition was measured. On the basis of this result, the expression of the alkaline protease from said transformed E. coli was low as known previously.

In order to solve the above problem, the present inventors cloned the recombinant plasmid vector pSBCm into Vibrio sp. As a result, however, the stability of the recombinant plasmid vector pSBCm was low. Therefore, in order to raise the stability of the said recombinant plasmid vector, the par gene that is known as being of the function of improving the stability of plasmid vector itself was inserted into the said recombinant plasmid vector pSBCm to construct the recombinant plasmid vector pSP1. As a result, the stability of the recombinant plasmid vector pSP1 was 40% higher than that of the recombinant plasmid vector pSBCm without the par gene.

The strain Vibrio metchnikovii KS1 that was deposited with the Korean Culture Center of Microorganisms, Seoul, Korea with an Accession Number of KFCC-10141 on Dec. 15, 1998, used in the present invention, is produced by treating Vibrio metchnikovii RH 530 N-4-8 with N, N-nitrosoguanidine(NTG) as a mutagen, and that was deposited with the Korean Culture Center of Microorganisms with an Accession Number of KFCC-11030 on Feb. 23, 1998. As a result, the amount of the alkaline protease produced from the V. metschnikovii KS1 was twice as high as that of the strain V. metschnikovii RH 530 N-4-8.

Also, the strain E. coli Top10F′ containing the recombinant plasmid vector pSBCm of the present invention was deposited with the Korean Culture Center of Microorganisms on Dec. 15, 1998, as Accession No. KCCM-10142.

Hereinafter, a more detailed description on the specific effect of the present invention will be given by the following Examples, without limiting the spirit and scope of the invention.

### EXAMPLES

### Example 1

Construction of the Recombinant Plasmid Vector pSBCm Comprising the Alkaline Protease Gene Vapk

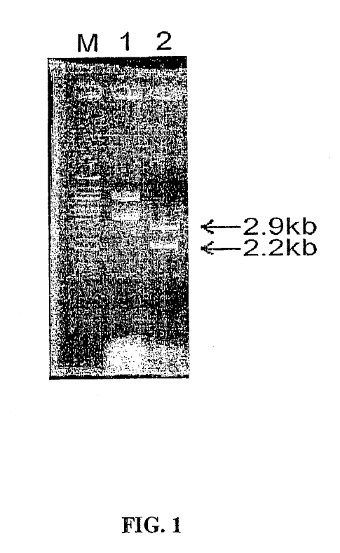

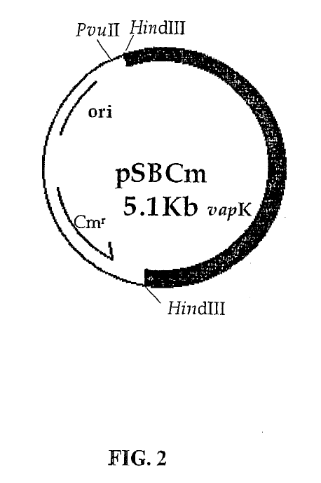

After culturing Vibrio metschnikovii the KS1 in a LSC medium with the composition of Table 1 at 30° C., the grown cells were harvested and disruptured. The disruptured solution was centrifuged at 6,000 rpm and the supernatant was collected. The chromosomal DNA of Vibrio metschnikovii KS1 was purified from the supernatant and partially digested with Hind III. The fragment was inserted into a vector pKF3 to clone 2.9 kb alkaline protease gene. The resulted recombinant plasmid vector was designated as pSBCm. The recombinant plasmid vector pSBCm was digested with Hind III and subjected to electrophoresis on 1% agarose gel. After electrophoresis, the agarose gel was stained with ethidium bromide as a staining agent. As a result of the above agarose gel electrophoresis, it was verified that the alkaline protease gene was cloned into a plasmid vector. (FIG. 1 and 2)

TABLE 1
Composition of LSC medium

| Composition of LSC medium | Amount (g/L) |
|---|---|
| Tryptone | 10 |
| Yeast extract | 5 |
| Sodium chloride | 10 |
| 1 M Sodium carbonate buffer, | 100 (ml/L) |
| pH 10.5 | |

### Example 2

Construction of the Recombinant Plasmid Vector pSP1 Comprising the Alkaline Protease Vapk Gene and the Par Gene

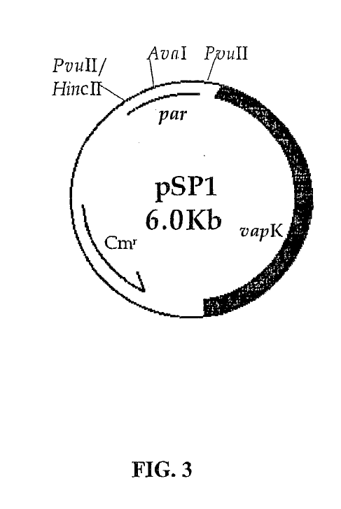

For the purpose of increasing the stability of the recombinant plasmid vector pSBCm, the par gene was cloned into a region between PvuII and HincII recognition sites of pSBCm to prepare the recombinant plasmid vector pSP1 with the alkaline protease gene vapk and the par gene (FIG. 3).

### Example 3

Transformation of E. coli With the Recombinant Plasmid Vector Comprising an Alkaline Protease Gene

The recombinant plasmid vectors pSBCm and pSP1 comprising the 2.9 kb alkaline protease gene of example 1 or example 2 was transformed into E. coli HB101.

After culturing the strain E. coli HB101 in a LB medium, 100 mM calcium chloride was added into the culture broth of E. coli HB101. The mixture was stored at 0° C. for 15 minutes and centrifuged to harvest E. coli HB101. The precipitated cells were suspended into a 100 mM calcium chloride solution and stored at 0° C. for 15 minutes. The recombinant plasmid vector pSBCm was added into the suspended solution and stored at 0° C. for 1 hour. The suspended solution was heated at 42° C. for 90 sec and stored at 0° C. for 5 minutes. After adding 1 ml of LB medium into the suspended solution, the solution was plated on LB agar medium containing 34 μg/ml of chloramphenicol and incubated at 30° C. for 24 hours, and the colonies grown were selected.

### Example 4

Transformation of Vibrio metschnikovii With the Recombinant Plasmid Vector Comprising the Alkaline Protease Gene

An electroporation method was used for the transformation of Vibrio metschnikovii KS1 with the recombinant plasmid vector comprising the alkaline protease gene.

After culturing the Vibrio metschnikovii KS1 in a LB medium at 30° C., the grown cells were harvested by centrifugation at 6,000 rpm, 4° C. for 10 minutes and suspended in a H-buffer solution consisting of 200 mM sucrose, 1 mM HEPES and 10% glycerol in the volume ratio of 20:1. After repeating the above process twice, and the cells were resuspended into a 80 μl H-buffer solution. The recombinant plasmid vector was added to the suspended solution and the solution was poured into a 0.2 cm cuvette. The electric power of 1,500V, 10 μF and 200Ω was applied to the cuvette using Gene Pulser II made by Bio-Rad Co. The 600 μl of LB medium was added into the electric power-treated solution and incubated at 30° C. The cultured medium was plated on the LB agar medium containing 25 μg/ml of chloramphenicol and the colonies grown were selected.

### Example 5

Assay of the Stability of the Recombinant Plasmid Vector Comprising the Alkaline Protease Gene Within the Transformed Vibrio sp.

Stability of the recombinant plasmid vectors with or without the par gene was assayed and compared. Transformed Vibrio metschnikovii pSBCm and the transformed Vibrio metschnikovii pSP1 were individually seed-cultured in a LB medium containing 25 μg/ml of chloramphenicol for 18 hours and growth-cultured in a LB medium without chloramphenicol. During the growth culture, the cultured broth was sampled several times at a certain interval and each sample was immediately plated on each LB agar medium with or without 12.5 μg/ml of chloramphenicol. After incubating the plated media at 30° C. for 20 hours, the number of colonies was measured (Table 2). On the basis of the number of grown colonies, the stability of the recombinant plasmid vector may be calculated by the following equation:

Stability of plasmid vector=(A/B)×100

A: the number of colonies grown on LB agar medium containing 12.5 μg/ml of chloramphenicol; and

B: the number of colonies grown on LB agar medium without chloramphenicol.

TABLE 2
The stability of the recombinant plasmid vector within the transformed
strain Vibrio metschnikovii pSBCm and Vibrio metschnikovii pSP1

| | | | | Stability of | |
|---|---|---|---|---|---|
| | Growth (O.D. 600 nm) | | | plasmid (%) | |
| | | pSBCm | pSP1 | pSBCm | pSP1 |
| Time (hr)/ | | within | within | within | within |
| generation^{a} | Vibrio sp. | Vibrio sp. | Vibrio sp. | Vibrio sp. | Vibrio sp. |
| 6/4.5 | 2.41 | 2.16 | 2.25 | 51.72 | 64.83 |
| 12/18 | 4.27 | 3.66 | 3.32 | 37.72 | 62.38 |
| 18/27 | 5.31 | 4.00 | 3.42 | 24.56 | 41.99 |
| 27/40.5 | 5.98 | 4.41 | 4.04 | 22.00 | 37.07 |

^{a}One generation time is approximately 40 minutes.

### INDUSTRIAL APPPLICABILITY

According to the present invention, the method for improving the stability of the recombinant plasmid vector comprising the vapk gene encoding an alkaline protease can be effectively used in the production of the alkaline protease using a V. metschnikovii strains.

What is claimed is:

1. A recombinant plasmid vector pSP1 in which the par gene is cloned into a recombinant plasmid vector pSBCm (KCCM-10142) comprising the vapk gene encoding an alkaline protease to improve the stability of plasmid vector itself.

2. A method for producing the recombinant plasmid vector pSP1 of claim 1, by inserting the par gene into between PvuII and HincII recognition sites of a recombinant plasmid vector pSBCm (KCCM-10142) comprising the vapk gene encoding an alkaline protease derived from Vibrio sp., wherein the par gene has the function of improving the stability of the plasmid vector itself.

3. A transformed host cell comprising the recombinant plasmid vector pSP1 of claim 1.

4. The transformed host cell according to claim 3, wherein said host cell comprises Escherichia coli or Vibrio metschnikovii.

5. A method for producing an alkaline protease, VapK, using the transformed host cell of claim 3 or 4.
